(19) United States
(12) Patent Application Publication
(10) Pub. No.: US 2011/0056493 A1
(43) Pub. Date: Mar. 10, 2011

(54) Sleep apnea vapor inhaler adapter

(76) Inventors: Kim Iola Miller, Medford, OR (US); Benjamin Cody Smith, Jacksonville, OR (US)

(21) Appl. No.: 12/584,655

(22) Filed: Sep. 10, 2009

(51) Int. Cl. A61M 16/10; A61M 16/16

(52) U.S. Cl. 128/203.16

(57) ABSTRACT

The sleep apnea adapter has many different usages. The usages include medications like nebulizers and “Vicks” to be inhaled. It can also be used with aroma therapies. I.E, Peppermint, lavender, cedar, and many other soothing oils or pastes. The application for this will sooth the patient whatever the case may be.

With the ability to distribute through multiple passage ways the patient is able to control the amount of vapor they desire. The air control can be a valve that can be twisted multiple different ways for the amount of vapor to air inhaled.

### FIELD OF THE INVENTION

The present invention relates to current sleep apnea treatment systems as typically used in the home, hospital, and many other locations for personal and or professional medical use. Our adaption incorporates into all existing sleep apnea systems on the market.

### BACKGROUND OF THE INVENTION

This inline medication adapter connects to all existing sleep adapting machines. Current sleep apnea machines forces air into the sinus cavities or air ways while the user sleeps. This adaption allows the user to inhale fragrances and or medications to be delivered directly into the sinus cavity or air ways via the unit. The unit can also heat to vaporize the medications for better effect. For certain prescription such as nebulizer treatments the unit will apply the medication in the same effect as a current nebulizer machine throughout the users sleep.

### SUMMARY OF THE INVENTION

The sleep apnea adapter will be made from plastic and or any other known composite materials. The unit may be one air way passage or may have multiple air passages for vapor amount control. It will be able to be easily removed by unthreading, unclipping, un-clicking, and or pressure released for cleaning or changing parts. It will house liquid, paste, and or any other vaporized medication or substance the patient, prescriber, and or user sees fit. The unit can be positioned either directly into the facial unit or inline between the machine and the face mask.

The sleep apnea adapter may have a threaded lower cavity or lower cap for the placement of direct medications and or have an insert tray in the lower cap/cavity for premade slot inserts medications. This adapter may have a lower cavity but also may be a direct line insert for cartridge medications. The system also may have an air bypass system in the event that something interferes with the air flow. The unit may also include a safety cage or screen to stop any larger or small objects from passing through the system and into the user's airway.

### BRIEF DESCRIPTION OF THE DRAWINGS

FIG. 1 shows the unit as a two or more piece unit. The bottom of the unit is removable by unthreading it, or by ways such as lock tights systems. This unit may be equipped with a troth or bowl to apply pastes or liquids. The troth or bowl is located in the bottom cap of the unit for ease in refill, or ease of cleaning. This same bottom cap may be equipped with a card slot or insert tray for premade inserts. The inserts may be put into the trays and when the lid is shut they will not move for safety.

FIG. 2 shows the sleep apnea adapter unit in its basic form. It shows the unit as a two piece adapter that has guard rails, card slots, or tray sides to insert a vapor card. Both sides of the unit are beveled to press into the existing sleep apnea system. This model is intended to connect directly to the face mask without much size difference then a swivel adapter. Though the sizes may vary this figure is small. The card slides into the tray on the hose side rather then the mask side and can be pressed together or could have threads, quick connect or any other means of connection. This unit may not have trays for cards, cartridges, or tablets. If fitted properly the unit may allow the insert to have free play within the tube, but not allow the insert to move down the tube or into the mask.

FIG. 3 shows the unit in a two chamber form. The unit uses a two way valve to control the amount of vapor the patient desires. The unit may have different types of control valves similar to a garden hose, but could also include other forms of control such as twist type.

FIG. 4 shows a bottom cap that can connect to figure one that has a built in heating unit for vaporization of chosen inhalant. The system may also be able to include a sealed vapor bottle that can be heated to vaporize oils, or to apply prescription nebulizer treatments for COPD or any other liquid prescriptions.

FIG. 5 shows the same unit as FIG. 2 with a detailed description of a cartridge insert. It shows the trays or rails and how the card may be fit to insure that the card/cartridge will fit and not move for safety.

FIG. 6 shows a typical sleep apnea face mask with our cartridge insert. As you can see the cartridge insert adapter is small and may also include swivels on either end.

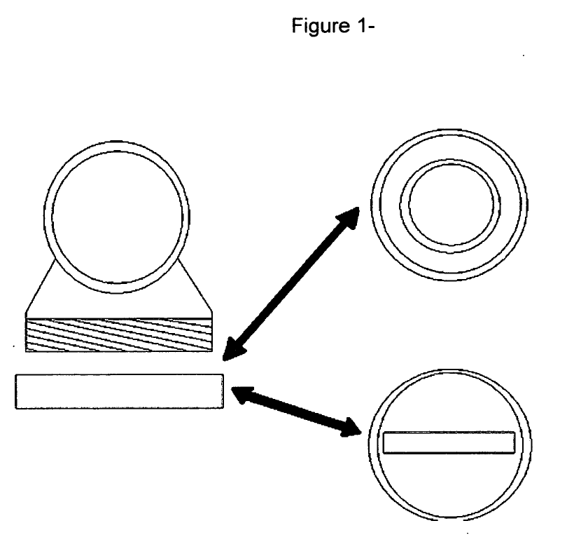

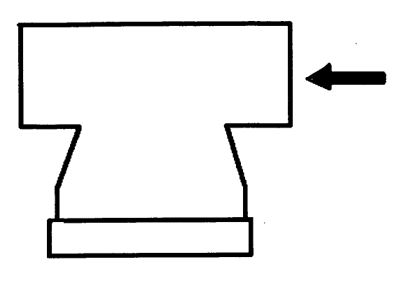

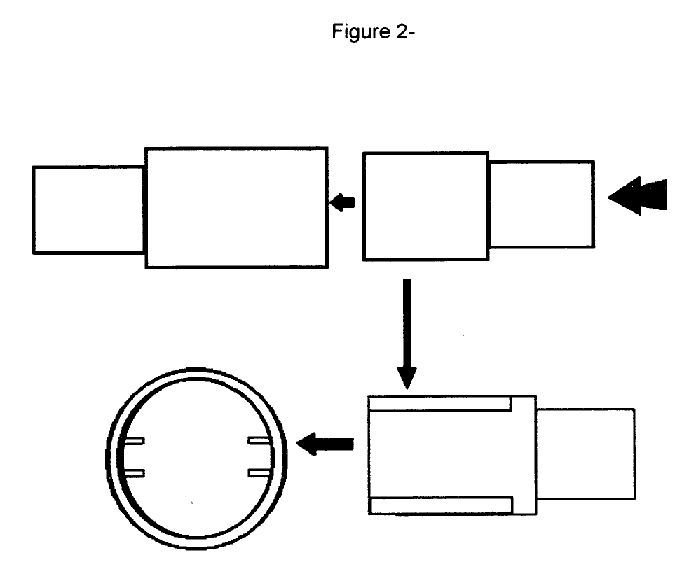

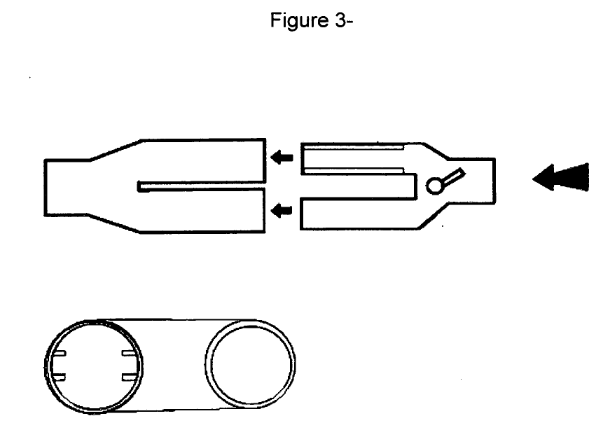

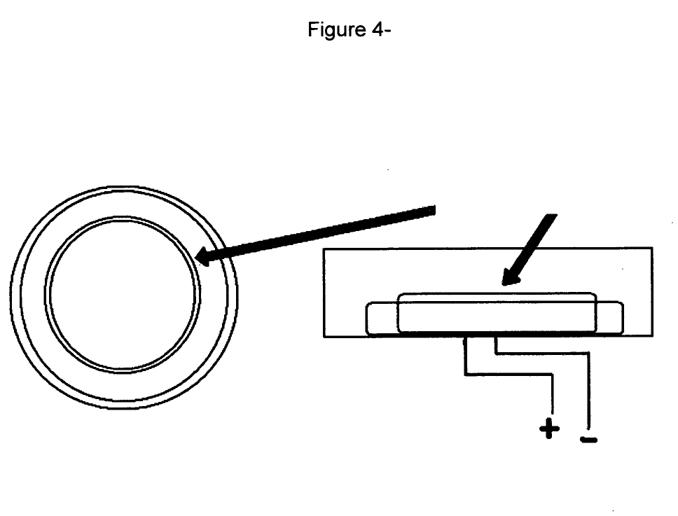

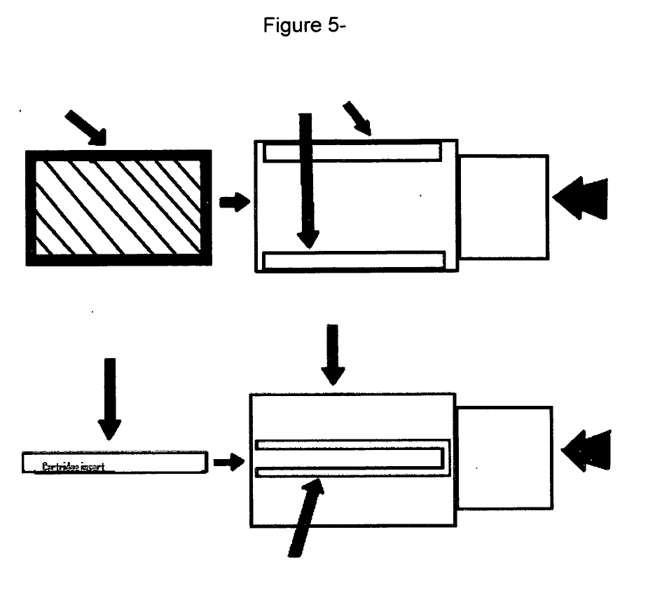

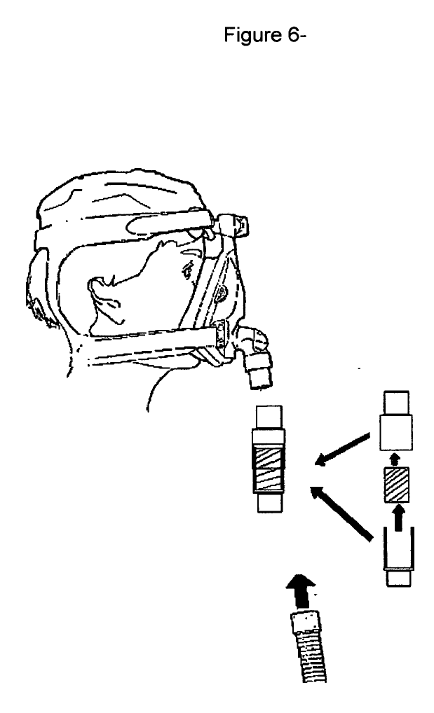

### DETAILED DESCRIPTION

The sleep apnea adapter maybe two pieces or more. It will be removable and slips into any existing current sleep apnea machine. Its usages are for doctor prescribed vapor or liquid medications, over the counter common products such as “Vicks or Eucalyptus”, or any other scented fragrances/aroma therapies for inhalation use. The adapter uses current air flow through a cartridge, around a cartridge, or through passage ways utilizing the fragrances and or medications and distributing through existing sleep apnea devices.

The inline adapter may be a straight line with built in cartridge tray or tablet tray. The trays insure proper distribution of whatever substance is within, and insure that the substances do not disrupt airways. The trays will house cartridges or capsules in a tight enclosed area like rails not allowing the trays to ajar or move.

The unit may incorporate air passage ways to distribute the medications in various ways. The adapter may include emergency or secondary passage ways in the event of blockage. The adapter may include a small power/electric operated heating unit. The heating unit will increase vaporization and may be used for other applications such as nebulizers.

The unit may be assembled by pressure connect, quick connect, threaded, and or any other air tight sealing system.

The unit may be directly incorporated into the nasal cavity/air way mask for direct distribution, or could be at the beginning of the line for larger quantities of medication to be distributed. This system will include various adaptable components to fit various adaptable sleep apnea machines.

The unit may include a safety cage or door trap system to block any foreign objects from passing through or blocking air ways.

The unit may include multiple air passage ways for variant vapor control or emergency air bypass system.

1. I claim the rights to an inline inhalant or vapor distributer to adapt to any existing sleep apnea machine for the purpose of distributing any vapor medication or vapor inhalation.

2. I claim the rights to an inline inhalant or vapor distributed adapter that delivers any form of vapor substance from a container with a troth, bowl and/or cartridge with guides or slots for an exact fit for safety.

3. I claim the right to an inline inhalant or vapor distributed adapter with multiple air passage ways for regulation of the amount of vapors or safety bypass in the event of blockage.

4. As in claim 1 the adapter will have various ways of construction such as threaded, twisted, locked, or press locked.

5. As in claims 1 and 2 the adapter may include a heating unit for vaporization or humidifying substances.

6. As in claims 1, 2 and 3 the unit may be constructed from many different materials like plastic or metal and varies colors.

7. As in claims 2 and 3 the unit will have built in trays or slots for the use of cartridges, tablets, or any other premade inserts.

8. As in claim 1 the unit may be built in many different sizes or shapes to fit any existing sleep apnea machines on the current market or future market.

9. As in claim 1 the unit may include many different connecters to adapt to different existing sleep apnea machines on the current market.

10. As in claims 1, 2, and 3 the unit can be used for the delivery of any prescriptions by doctors, or normal over the counter substances such as “Vicks” or aroma therapies in any solid, paste, or liquid form.

11. As in claims 2 and 3 the unit may be constructed in one piece or in multiple pieces made in any varies colors. The unit may be disassembled for cleaning or changing parts for different uses.

12. As in claim 1 the unit may include a safety screen or cage for catching anything that may become lose or ajar that may block air ways.

13. As in claim 1 the unit may include an air bypass for safety in the event of airway blockage.

14. As in claim 1 the unit may include swivels on either side.

15. As in claims 1 and 2 the unit may include a sealed bottle or bag for controlled vapor release by heater unit.

16. As in claim 1 the unit may include twisted fins for air control or air way direction.
